(19) United States
(12) Patent Application Publication
(10) Pub. No.: US 2004/0026229 A1
(43) Pub. Date: Feb. 12, 2004

(54) Method and device for the mask-free production of biopolymers by means of a light diode array

(76) Inventors: Heinz Eipel, Bensheim (DE); Markus Beier, Heidelberg (DE)

Correspondence Address: Keil & Weinkauf, 1101 Connecticut Avenue NW, Washington, DC, 20036, US

(21) Appl. No.: 10/168,214

(22) PCT Filed: Dec. 27, 2000
(86) PCT No.: PCT/EP00/13295

(51) Int. Cl. G01N 33/53

(52) U.S. Cl. 204/157.6; 204/157.68; 435/968

(57) ABSTRACT

The invention relates to a process and a device for the light-controlled synthesis of biopolymers on surfaces. In this process, patterns of individual sequences (20) are produced by the imaging of an arrangement (1) of electrically individually controllable light diodes (2) onto the surfaces.

The invention relates to a process and a device for the mask-free preparation of biopolymers which are synthesized, for example on a slide, by means of an exposure sequence.

Up to now, mask sets for each individual chip have been necessary for light-controlled DNA chips synthesis. U.S. Pat. No. 5,412,087 discloses a regionally addressable immobilization of oligonucleotides and other chemical polymers on surfaces. According to this process, it is proposed that substrates with surfaces which contain components having thiol groups and photoactively removable protective groups can be used to produce fields of immobilized anti-ligands, such as, for example, oligonucleotides or other biopolymers. The fields are used to detect the presence of complementary nucleic acids in a liquid sample. The regionally addressable irradiation of predefined regions on the surface permits the immobilization of oligonucleotides and other biopolymers in the activated regions of the surface. Irradiation cycles on different surface regions of the surface and immobilization on various anti-ligands allow the formation of an immobilized matrix of anti-ligands in certain positions on the surface. The immobilization matrix of the anti-ligands allows a simultaneous thorough search of a liquid sample for ligands which have a very high affinity for certain anti-ligands in the matrix.

In the process proposed here, the surface of the substrate body is irradiated through a mask with a light source which emits a wavelength in the range between 280 and 420 nm, where only certain preselectable regions can be selected for irradiation with each individual mask.

U.S. Pat. No. 5,744,305 relates to materials applied to a support in the form of fields. These are used for a synthesis strategy for the production of chemically divergent substrates. Molecular groups having photoactive protection action are used in order to achieve light-controlled chemical synthesis processes proceeding regionally in parallel. Binary masking techniques are employed in the context of a working example. In the process disclosed, various chemical components are synthesized in parallel using a masked radiation source or by means of an activator. The exposure pattern defines which regions of the slide are prepared for a chemical reaction. Here, too, the masking technique is employed in order to select different regions to be exposed in each case on the slide.

U.S. Pat. No. 5,143,854 relates to a process for the photolithographic synthesis of polypeptides and to a search process. In this process, polypeptide fields are synthesized on a substrate in which photoactive groups are applied to the surface of a substrate which expose certain regions of the substrate to light for the activation of the regions. To the regions activated in such a way, an amino acid monomer having a photoactive group is applied, the activation and addition steps being repeated until polypeptides of the desired length and sequence are synthesized. The resulting field can be used for the selection of those peptides which are able to bind to a receptor.

In U.S. Pat. No. 5,143,854, in addition to the masking method already discussed, it is proposed to employ a diode light source for the exposure and to expose the substrate to be exposed according to the sections to be exposed. In this procedure, a complicated mechanical control mechanism is necessary in order to align the substrate as accurately as possible corresponding to the regions to be exposed by the light-emitting diode. This mechanical alignment must be carried out anew each time for each new field to be exposed.

The control mechanism for achieving the set positions mentioned makes very high demands on the manufacturing accuracy.

In addition to the masking of the biopolymer regions to be exposed on the slide, it is disclosed in WO 99/42813 to expose DNA sequences or polypeptides or the like by means of an arrangement having controllable micromirrors in each case, where the micromirrors form a coherent field which is composed of electronically addressable individual micromirrors. A common light source is assigned to this. The biopolymers situated on the slide are activated in certain patterns, the monomeric units which are supplied sequentially in each case being coupled to the controlled regions. This process is continued until all elements of a two-dimensional field on the substrate have reacted with the monomer desired in each case. The micromirror field can be controlled, e.g. in combination with a DNA synthesizer, such that the image sequence is coordinated by the micromirror field with the liquid sample applied to the slide.

In the masking processes shown, photoactive monomeric units or photoactive surfaces are used in order to make possible a site-directed synthesis. The action of light is used to remove the photoactive groups of the monomeric units or surfaces in order for a synthesis or an immobilization step then to be able to take place in these positions of the action of light. In order to bring the light exclusively onto the site needed in the respective synthesis or immobilization step, masks are employed or micromirror fields are controlled.

If, for example, in the case of light-controlled oligonucleotide synthesis n nucleotides are linked in an n-mer sequence from an ensemble of four different bases, 4×n masks are needed. If a light-controlled synthesis of peptides with a sequence length of n and an ensemble of 20 amino acids is to be carried out, 20×n masks are needed. A mask set of this type must not only already be provided in advance, but also has to be adjusted very accurately during the exposure. This is accompanied by a considerable technical outlay, so that the masking process is not worthwhile for small series for the reason that in each new synthesis a new mask set has to be provided.

The modulable light source disclosed in U.S. Pat. No. 5,143,854 allows the translatory adjustability of the slide with a corresponding mechanical outlay. A disadvantage is furthermore the fact that the exposure can only be carried out sequentially and not in parallel.

The invention is based on the object of making available a process for the synthesis of biopolymers, which simplifies and makes mask-free exposure and activation of individual regions of a biopolymer-accepting slide.

According to the invention, this object is achieved by the features of claims 1 and 15.

The advantages accompanying the solution proposed according to the invention are of varied nature. Using very simple means, it is possible to synthesize arrays of biopolymers such as, for example, oligonucleotides and peptides. Furthermore, biopolymers can be immobilized in a light-controlled manner; no masks are needed and working steps used for their preparation and setting up are completely unnecesssary. With the abolishment of the masks, the adjustment sequences connected therewith are also completely unnecessary. Neither complicated and expensive mechanical displacement tables for the control of the individual synthesis sites nor complicated positioning equipment are needed. The exposure steps are all able to proceed in parallel; furthermore, as a result of the masks being unnecessary the synthesis of individual or small series can be carried out extremely economically by means of the process proposed according to the invention.

In an advantageous embodiment of the process according to the invention, nucleotides and peptides can be synthesized on surfaces. In addition, biopolymers also can in each case be immobilized on the surfaces. Biopolymers are understood as meaning, for example, nucleic acids, their analogs (e.g. PNA, LNA), amino acids, peptides, proteins, carbohydrates, as well as combinations of these. The switching-on and switching-off of the light diodes of the light diode array of 9, 16, 25 or up to 100 or even more light diodes is preferably automatically controlled by means of an arithmetic unit. The arithmetic unit contains the radiation arrangements desired in each case in stored form.

By means of the arithmetic unit, the exposure time may also be input during which the individual light diodes irradiate selected regions of a slide. Preferably, those light diodes are used which emit an energy-rich radiation in the UV range. For shortening the synthesis process by reducing the activation or immobilization times, exposure processes can be carried out simultaneously by means of the light diode array containing a number of light diodes. A sequential order of exposure processes can in addition also be set up.

The simultaneity of the control of a number of light diodes of the light diode array can be realized, for example, by the control of the light diodes via the parallel interface of a computer. The parallel carrying-out of exposure cycles taking into consideration the sequence of individual exposure times stored in the arithmetic unit shortens the synthesis of biopolymers considerably. The substrate for the biopolymers to be synthesized thereon is situated in a feed arrangement below a light-transmitting region. The feed arrangement can be configured, for example, as a flow chamber in which the chemicals needed for the synthesis to be carried out can be supplied sequentially. The respective sequences for the individual fields of the array to be synthesized are first input into the controlling computer. Using an appropriate program, the computer, according to these specifications, controls the individual light diodes in the light diode array correlated with sequential and cyclic supply of the individual monomers.

Preferably, the exposure takes place spatially separate from the chemical synthesis in order to exclude any external interfering effects during the exposure.

In addition to the pro sequence of biopolymers to be synthesized individually, the sequential order of immobilization places for freely selectable biopolymers can also be stored in the arithmetic unit.

By means of the device proposed according to the invention, a parallel light-controlled synthesis or immobilization of biopolymers can be achieved by computer-supported parallel control of individual light diodes without masks being necessary. Preferably, the light diodes are designed as light diodes emitting light in the UV wavelength range. For the geometric scaling of the biopolymer array, it is possible, for example, to carry out an optical imaging of the light diode array on the desired scale. For this, accordingly suitable optical devices are employed.

The invention is illustrated in greater detail below with the aid of the figure:

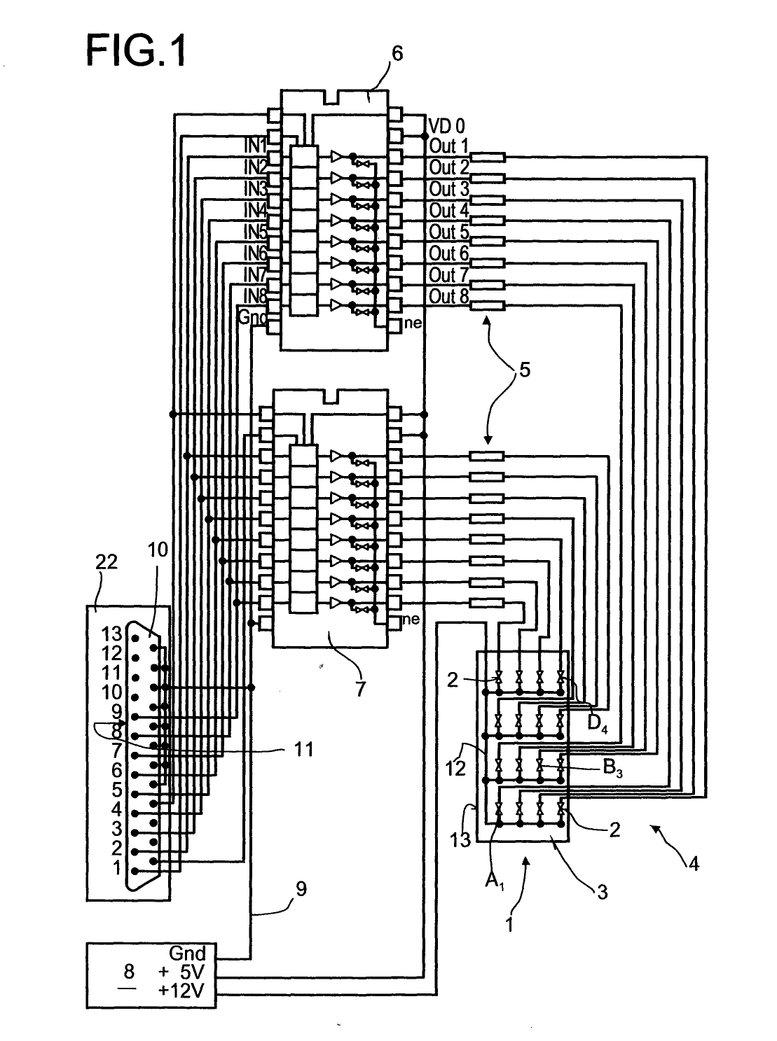

FIG. 1 shows a field of, for example, 4×4 individually controllable light diodes with the electrical control lines,

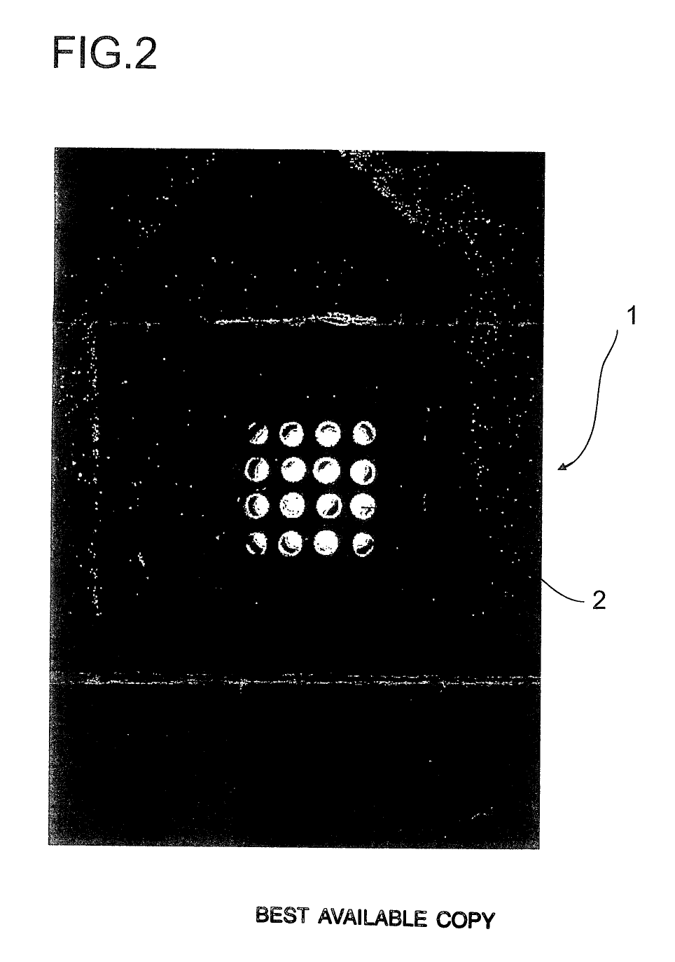

FIG. 2 shows a fluorescent image of an oligonucleotide array, synthesized using a 4×4 light diode array after hybridization,

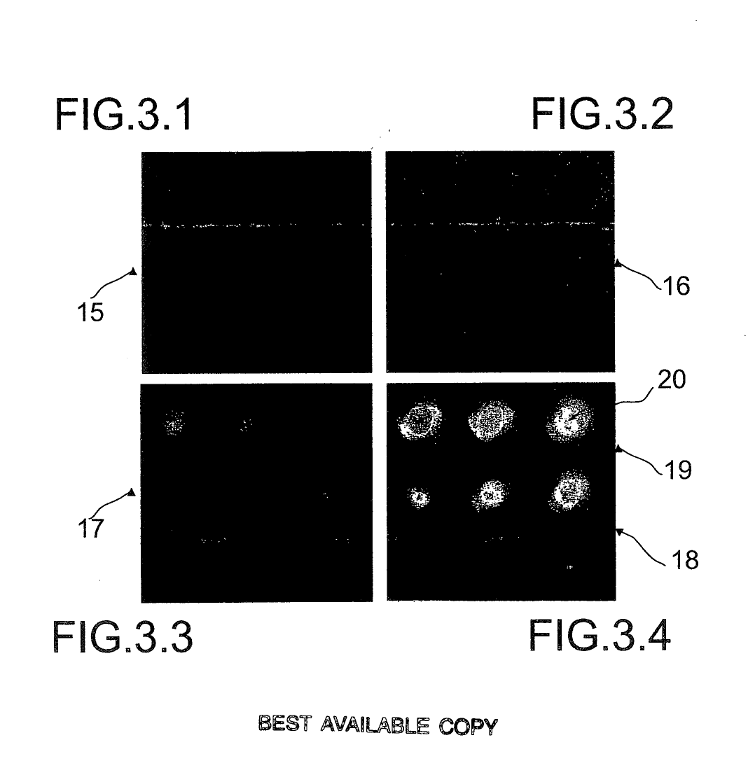

FIG. 3 shows four surface fluorescence images in four different sensitivity stages for the determination of an adequate exposure time using a 3×3 light diode array,

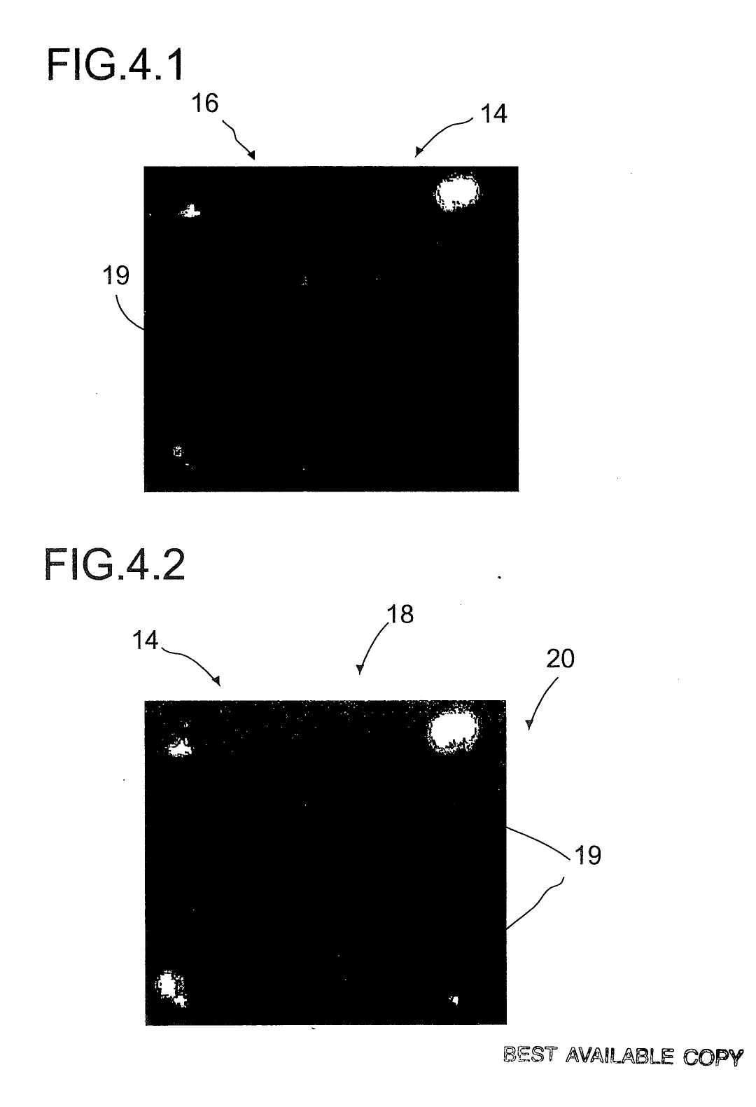

FIG. 4 shows the synthesis of a sequence on a chip surface with the aid of a 2×2 light diode array.

In FIG. 1, the top view onto a field having, for example, 4×4 individually controllable light diodes and the associated control electronics are shown by way of example.

FIG. 1 shows in schematic representation a light diode array 1 which is below a slide 12 or below a chip surface 19. The arrangement drawn in FIG. 1 is a light diode arrangement 1 which contains 16 individually electrically controllable light diodes 2; of the light diodes 2 shown, the light diodes A1, B3 and D4, which can be controlled, for example, for one of sixteen DNA sequences, are shown in greater detail.

Any control of a light diode 2 of the light diode array 1 is carried out via separate control lines 4, the individual light diodes 2 being connected to the supply section 8. Furthermore, the individual light diodes 2 of the light diode array 1 are in each case connected to resistances 5, from which further lines extend to memory cells 6 and 7. The memory cells 6 and 7 for their part are controlled by means of a parallel interface 10 provided on an arithmetic unit 22.

In the computer 22 which is only shown here schematically with its parallel interface 10, it is possible to store in various data files, for exmaple, the DNA sequences 20 and also the exposure times necessary for the removal of the individual photolabile protective groups or alternatively the sequential order of immobilization places. Furthermore, those chemicals needed for the synthesis of biopolymers such as, for example, oligonucleotides or peptides can be provided by the arithmetic unit 22, where these, depending on the sequence to be treated, react at exactly specifiable exposure sites after the labile photoprotective groups have been removed there. By means of the correlation of the sequences with the chemical supply and the associated exposure sites, it is possible by the use of the parallel port 10 of the arithmetic unit 22 to carry out a simultaneous exposure of a number of exposure sites.

The light-controlled synthesis takes place, for example, in a feed device which can be designed, for example, as a flow cell. The flow of the chemicals needed for the synthesis flowing through the flow cell is controlled by a DNA synthesizer, for example, of the arithmetic unit 22. In this, the DNA sequences 20 are present in data files, for example, in stored digital form.

In order, within a synthesis cycle taking place in a flow chamber, to define in which position which of the four nucleotide units—of deoxyadenosine, deoxythymidine, deoxyguanosine and deoxycytidine—is to be condensed, photolabile protective groups on the substrate support must be removed at specified times. The removal of the photolabile protective groups is necessary, because only after their removal can a synthesis and the construction of a DNA oligomer be achieved. The exposure of the substrate support at the sites at which the photolabile protective groups are to be removed is carried out by the light diode arrangement 1 according to FIG. 1. The individual light diodes 2 of the light diode arrangement 1 are preferably designed as individually electrically controllable light diodes 2. These emit a very energy-rich radiation, preferably in the ultraviolet range, having a wavelength of preferably 360 nm. However, light diode arrangements 1 can also be used in which individual light diodes 2 are contained which emit a radiation of other wavelength, which is different than the UV range. The optimum wavelength of the light diodes 2 is to be coordinated with the photochemistry used.

By means of the control of the individual light diodes 2 of the light diode arrangement 1, it is defined in which position of the substrate support photoprotective groups are removed in order to make possible the addition of nucleotide units to be coupled. For this, in FIG. 1, by way of example, three light diodes 2 are singled out which are designated by A1, B3 and D4. The emitted energy-rich radiation of the light diodes 2 designated by A1, B3 and D4 preferably strikes in the positions of the substrate support of the feed arrangement and causes the removal of the labile photoprotective groups at the thus exactly defined sites. The exposure time can be different depending on the substrate applied to the support, also depending on the sequence to be produced. The different exposure times which are to be adhered to by the light diodes with respect to their switching-on time can likewise be deposited in a data file of the arithmetic unit 22 and in this manner incorporated into the proposed exposure process.

By means of the control of the light diodes 2 corresponding to the positions A1, B3 and D4, a removal of the protective groups now takes place in these positions of the substrate support, such that a chain lengthening is also only achievable at these well-defined sites within this synthesis step on the substrate support. During, for example, a subsequent synthesis step, it is possible to carry out a removal of the photolabile protective groups on the substrate, for example, in the positions A4, B2 and D1 such that, after expiry of the exposure time needed for the removal of the protective groups, a chain lengthening with making available of a monomeric unit to be fed through the flow chamber can only take place at these sites. By means of the procedure outlined here, the light diode arrangement 1 is employed as a field of individual light sources without an individual mask set for each substrate support or each chip surface 19 being necessary. By means of the computer-supported separate control of individual light diodes with respect to time of action of the exposure and preselection of the exposure sites, depending on the sequence data file deposited in the computer 22, small series can also be synthesized advantageously.

The light diode arrangement 1 controlled by means of the arithmetic unit 22 takes over both the function of the exposure and that of masking of the region to be exposed, on account of which the necessity to reposition masks can be completely omitted. Inaccuracies in the mask repositioning during the synthesis after the masking process have in the past led to considerable quality deficiencies in biopolymer units synthesized in such a manner.

FIG. 2 shows a synthesized oligonucleotide array, synthesized using an array of 4×4 individually electrically controllable light diodes 2. In addition to the configuration of a light diode array 1 shown here, this can also arbitrarily contain many, for example 25, 400 or up to several thousand individual radiation sources in the form of light diodes, where it can be left open whether these can be arranged as a square, rectangle, ring or alternatively circle. The array depicted in FIG. 2 is a fluorescence image which was obtained by hybridization using a fluorescence-masked complementary strand probe.

FIG. 3 shows in overall view four surface fluorescence images, in each case photographed using four different sensitivity stages. For the determination of an adequate exposure time, a 3×3 light diode array 1 was used. The four images show the same array, photographed in four different sensitivity stages of the detecting scanner.

While no signals are contained in the surface fluorescence images 14, 3.1 and 3.2 photographed using low sensitivities 15, 16, these are clearly discernible using higher sensitivity stages 17 or 18 in the surface fluorescence images shown in FIGS. 3.3 and 3.4. The intensity of the signals is proportional to the efficiency of the cleavage of the labile photoprotective groups in the respective position on the substrate support 12 with a specifiable irradiation period. The irradiation was carried out with a period of 1, 3, 5, 7, 10, 13, 15, 20 and 30 minutes. The successful removal of the photoprotective group on account of the irradiation was visible by means of a covalent bonding of a Cy 5 phosphoramidite after irradiation had taken place.

FIGS. 4.1 and 4.2 render visible the construction of a sequence on a surface 19 using a light diode arrangement 1 containing 4 individual light diodes 2.

In the four positions 19 shown as lighter, the sequence d(CGCTGGAC) was constructed by means of light-controlled DNA chip synthesis on the surface fluorescence images 14, which have been photographed using different sensitivities 16, 18. For this, a light diode array 1 containing four individual light diodes 2 was used, which emit radiation in the ultraviolet wavelength range. The images shown in FIGS. 4.1 and 4.2 were photographed using different sensitivities of the scanner. Using a 2×2 UV light diode arrangement 1, a DNA chip synthesis of the sequence CGCTGGAC was carried out, which was hybridized with fluorescence-labeled GTCCAGCG with an exposure time of 10 minutes.

The depicted FIGS. 4.1 and 4.2 were obtained after hybridization with the complementary 5′-Cy-5-labeled probe after scanning in a fluorescence imaging unit.

List of reference symbols

| | |
|---|---|
| 1. Light diode array | |
| 2. Individual light diode | |
| 3. Field boundary | |
| 4. Control line | |
| 5. Resistance | |
| 6. Memory cell | |
| 7. Memory cell | |
| 8. Supply voltage section | |
| 9. Grounding | |
| 10. Parallel interface PC | |
| 11. Interface line 1 to 25 | |
| 12. Slide | |
| 13. Lateral edge | |
| 14. Surface fluorescence image | |
| 15. Sensitivity low. | |
| 16. Sensitivity higher | |
| 17. Sensitivity high | |
| 18. Sensitivity very high | |
| 19. Chip surface | A1 Light diode position |
| 20. Sequence d (CGCTGGAC) | B3 Light diode position |
| 21. Sequence position | D4 Light diode position |
| 22. Arithmetic unit | |

1. A process for the light-controlled synthesis of biopolymers on surfaces, which comprises bringing about selection and activation of regions on a solid support by the imaging of an arrangement (1) of electrically controllable light diodes (2).

2. A process as claimed in claim 1, wherein biopolymers are immobilized on slides (12,19).

3. A process as claimed in claim 1, wherein the light diodes (2) are individually controlled according to a data file of sequences (20) stored in a computer (22).

4. A process as claimed in claim 1, wherein the individual light diodes (2) emit energy-rich radiation in the UV range.

5. A process as claimed in claim 1, wherein the exposure of a number of regions is carried out simultaneously by means of the light diodes (2).

6. A process as claimed in claim 1, wherein the exposure is carried out sequentially.

7. A process as claimed in claim 1, wherein the control of the light diodes (2) of the light diode array (1) is carried out by means of the parallel interface (10,11) of an arithmetic unit (22).

8. A process as claimed in claim 1, wherein the substrate for the biopolymers to be synthesized thereon is situated in a feed arrangement under a light-transparent region.

9. A process as claimed in claim 8, wherein the chemicals needed for the synthesis are supplied sequentially and the exposure is carried out in the feed arrangement.

10. A process as claimed in claim 1, wherein the exposure takes place spatially separate from the chemical synthesis.

11. A process as claimed in claim 2, wherein the sequential order of the immobilization places in the computer (22) is stored in a data file.

12. A process as claimed in claim 1, wherein suitable optical imaging processes are used for the geometric scaling of the light diode array (1) onto the biopolymer array.

13. A device for light-controlled biopolymer synthesis on slides (12,19), having an exposure source, wherein an exposure arrangement (1) which consists of electrically controllable light diodes (2) is assigned to a slide (12,19).
